(19) United States
(12) Patent Application Publication
(10) Pub. No.: US 2007/0021438 A1
(43) Pub. Date: Jan. 25, 2007

(54) Itraconazole solution

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 10/570,497

(22) PCT Filed: Sep. 1, 2004
(86) PCT No.: PCT/CA04/01607

§ 371 Date: Mar. 3, 2006

(30) Foreign Application Priority Data
Sep. 5, 2003 (CA) 2440172

(51) Int. Cl. A61K 31/496

(52) U.S. Cl. 514/254.07

(57) ABSTRACT

A solution for oral administration comprising itraconazole, an alcoholic solvent, and an acid.

### BACKGROUND OF THE INVENTION

Itraconazole is a compound useful for treating fungal infections in mammals. Itraconazole is practically insoluble in water. This makes it difficult to, formulate itraconazole into solutions that are suitable for oral administration.

A solution for oral administration comprising itraconazole is sold in the United States and elsewhere under the tradename Sporanox™. The concentration of the solution is 10 mg itraconazole per mL, and the inactive ingredients in addition to water are hydroxypropyl-beta-cyclodextrin, propylene glycol, sorbitol, hydrochloric acid, sodium hydroxide, sodium saccharin, and flavours. The purpose of the hydroxypropyl-beta-cyclodextrin is to improve the aqueous solubility of itraconazole by forming an inclusion complex.
™—Registered Trademark

U.S. Pat. No. 6,340,698 discloses itraconazole solutions for oral administration that are free of cyclodextrins, and that comprise itraconazole, an acid, and one or more alcoholic solvents, wherein the concentration of alcoholic solvent is from 200 to about 800 mg per mL.

In the only example given in that patent, the acid used is concentrated hydrochloric acid at a level of 4 mg per mL, and the alcoholic solvent is propylene glycol at 520 mg per mL. The solution also comprises 660 mg per

mL of sorbitol solution 70%, which is comprised of 70% sorbitol and 30% water. The water content in the example is thus about 200 mg per mL.

It has subsequently been found that, for the solutions of U.S. Pat. No. 6,340,698, the stability of the itraconazole against chemical degradation is inadequate, such that the rate of degradation of the itraconazole is substantially greater than that of Sporanox™ solution.

The objective of the present invention is thus to enable solutions for oral administration comprising itraconazole at a concentration of 10 mg per mL that do not comprise a cyclodextrin and are more stable against chemical degradation than the solutions of U.S. Pat. No. 6,340,698.

### DESCRIPTION OF THE INVENTION

It has now been discovered that a solution that is free of cyclodextrin, that comprises itraconazole at a level of about 10 mg per mL, and that is more stable against chemical degradation than the solutions of U.S. Pat. No. 6,340,698, can be made by increasing the content of alcoholic solvent to above 800 mg per mL, reducing the water content to below 180 mg per mL, and using the minimum amount of acid required to keep the itraconazole dissolved without precipitation at room temperature.

The solutions of the present invention are thus cyclodextrin-free solutions comprising about 10 mg per mL itraconazole, an alcoholic solvent at a level above 800 mg per mL, an acid, and less than 180 mg per mL water.

In this specification, “cyclodextrin” will be understood to encompass any cyclodextrin derivative; “about” will be understood to mean within±10%; and “alcoholic solvent” will be understood to encompass any water-soluble mono-alcohol, diol or polyol that is a liquid at 25° C.

Preferred alcoholic solvents for use in the invention are propylene glycol, ethanol and glycerol. Most preferred is propylene glycol.

The acid will preferably be selected from hydrochloric acid, acetic acid and sulfuric acid, and will most preferably be hydrochloric acid. As aforesaid, the amount of an acid will preferably be the minimum needed to make the solutions stable against precipitation of the itraconazole.

The concentration of the alcoholic solvent will be above 800 mg per mL, will preferably be above 900 mg per mL, and will most preferably be about 1000 mg per mL.

The concentration of water will be under 180 mg per mL, will preferably be under 100 mg per mL, and will more preferably be under 25 mg per mL. The solution will most preferably be free or substantially free of water.

The pH of the solution will preferably be from about 1 to about 2, and most preferably from about 1 to about 1.5.

The solution will optionally contain other ingredients, in addition to itraconazole, an alcoholic solvent, an acid and water. For example, the solution will preferably contain a sweetener, such as saccharin and a flavouring ingredient.

The invention will be better understood from the following example which is intended to be illustrative of the invention, and not limiting.

### EXAMPLE 1

Solution was prepared comprising ingredients in the following amounts per mL.

| | | |
|---|---|---|
| Itraconazole | 10 | mg |
| Propylene Glycol | 1026. | mg |
| Concentrated Hydrochloric Acid | 1.7 | mg |
| Cherry Flavour | .3 | mg |
| Caramel Flavour | .1 | mg |
| Saccharin | 1 | mg |
| | 1039 | mg |

The ingredients were blended together at elevated temperature, and the resulting solution was then allowed to cool to room temperature. This solution was found to be stable against precipitation of itraconazole, and also to have stability against chemical degradation of the itraconazole comparable to that of Sporanox™ solution.

1. A cyclodextrin-free solution for oral administration comprising about 10 mg per mL itraconazole, an acid, and alcoholic solvent, wherein the concentration of alcoholic solvent is greater than 800 mg per mL and the amount of water is less than 180 mg per mL.

2. A solution of claim 1 comprising propylene glycol as alcoholic solvent.

3. A solution of claim 2 comprising propylene glycol as the sole alcoholic solvent.

4. A solution of claim 1 wherein the acid is selected from hydrochloric acid, acetic and sulfuric acid.

5. A solution of claim 4 wherein the acid is hydrochloric acid.

6. A solution of claim 1 wherein the amount of alcoholic solvent is greater than 900 mg per mL.

7. A solution of claim 1 wherein the amount of alcoholic solvent is about 1000 mg per mL.

8. A solution of claim 1 wherein the amount of water is less than 100 mg per mL.

9. A solution of claim 8 wherein the amount of water is less than 25 mg per mL.

10. A solution of claim 1 that is substantially free of water.

11. A solution of claim 1 having pH from about 1.0 to about 1.5.
